(19) United States
(12) Patent Application Publication
(10) Pub. No.: US 2010/0055036 A1
(43) Pub. Date: Mar. 4, 2010

(54) PET VISUALIZATION OF AMYLOID-ASSOCIATED NEUROINFLAMMATION IN THE BRAIN

(75) Inventors: Tetsuya Suhara, Chiba-shi (JP); Kazutoshi Suzuki, Chiba-shi (JP); Makoto Higuchi, Chiba-shi (JP); Ming-Rong Zhang, Chiba-shi (JP); Jun Maeda, Chiba-shi (JP); Bin Ji, Chiba-shi (JP)

Correspondence Address: SUGHRUE MION, PLLC, 2100 PENNSYLVANIA AVENUE, N.W., SUITE 800, WASHINGTON, DC, 20037, US

(73) Assignee: National Institute of Radiolotgical Sciences, chiba-shi, Chiba (JP)

(21) Appl. No.: 12/531,051

(22) PCT Filed: Mar. 12, 2008
(86) PCT No.: PCT/JP2008/055017

§ 371 Date: Oct. 13, 2009

Related U.S. Application Data
(60) Provisional application No. 60/906,183, filed on Mar. 12, 2007.

(51) Int. Cl. A61K 51/00; C07C 233/00

(52) U.S. Cl. 424/1.89; 424/1.65; 564/170

(57) ABSTRACT

The present invention relate to a method for monitoring a response to a therapy on a mammal having a neurodegenerative or neuroinflammatory disorder. According to a preferred embodiment, the method comprising the steps of:

a) imaging the mammal using a radio-labeled peripheral benzodiazepine receptor ligand;

b) administrating in the mammal at least one anti-amyloid or anti-neuroinflammatory agent;

c) imaging the mammal of step b) using a radio-labeled peripheral benzodiazepine receptor ligand; and

d) detecting the level of CNS neuroinflammation by the signals from the radio-labeled peripheral benzodiazepine receptor ligand.

The present application claims priority from the U.S. provisional application No. U.S. 60/906183, the content of which is hereby incorporated by reference into this application.

### TECHNICAL FIELD

The present invention relates to a longitudinal, quantitative assessment of neuroinflammation and anti-amyloid treatment in a subject with diseases associated with aggregated amyloid, especially Alzheimer's disease, enabled by PET.

### BACKGROUND ART

The diagnosis of Alzheimer's disease (AD) does not become definite unless neuropathologists examine the autopsied brain and score AD-characteristic amyloid lesions, which are known as senile plaques and neurofibrillary tangles and mechanistically implicated in neurodegenerative processes. Meanwhile, attempts to noninvasively visualize amyloid deposition in human brains using positron emission tomography (PET) have been made by developing imaging agents capable of reacting with amyloid fibrils (Sair et al., 2004; Nichols et al., 2006), among which N-[¹¹C]methyl-2-(4′-methylaminophenyl)-6-hydroxybenzothiazole ([¹¹C]6-OH-BTA-1, also known as Pittsburgh Compound-B) is the most intensively evaluated in human PET studies (Klunk et al., 2004; Price et al., 2005; Mintun et al., 2006; Engler et al., 2006). The ability of [¹¹C]6-OH-BTA-1 to detect amyloid in patients with mild cognitive impairment (MCI) (Price et al., 2005) and in a nondemented population (Mintun et al., 2006) has suggested the potential of this probe for identifying the AD pathology antecedent to the clinical onset. Such evidence, however, also leads researchers to question the applicability of [¹¹]6-OH-BTA-1 to antemortem staging of amyloid pathology and evaluation of candidate disease-modifying treatments in MCI and AD patients, as levels of radiotracer accumulation appear to plateau at an initial stage of the disease (Price et al., 2005; Engler et al., 2006). In addition, notable accumulation of this and other amyloid tracers in some amyloid-unrelated regions of human brains (Klunk et al., 2004; Shoghi-Jadid et al., 2002; Verhoeff et al., 2004) might arouse controversy over the specificity of this imaging technique for neurodegenerative pathologies. To efficiently exploit radioligands suitable for the purpose of establishing an early and sensitive marker of brain amyloidosis, or an objective measure of neuropathological severity in the progression of AD, preclinical screening of the candidate compounds by using in vivo systems is highly requisite. Such systems could also promote a proof-of-concept study on novel treatments (Scarpini et al., 2003) capable of suppressing neurotoxic amyloid aggregates.

There have been numerous lines of transgenic (Tg) mice that overexpress human mutant amyloid precursor protein (APP) causative of familial AD and recapitulate plaque pathology in AD brains (Hsiao et al., 1996; Sturchler-Pierrat et al., 1997). As shown by several investigations (Bacskai et al., 2003; Hintersteiner et al., 2005; Higuchi et al., 2005), use of fluorescent and MRI probes offers methodologies to capture brain amyloid in these animals. However, optical and MRI tracers need to be administered at a dose ranging from 0.1 to 1 μmol, which is much higher than that required for PET scans (0.1-1 nmol) and thus might influence the course of amyloid pathogenesis particularly in longitudinal multi-scan experiments.

### DISCLOSURE OF THE INVENTION

While improvements of both detection instrument and imaging agent to increase sensitivity of these modalities are ongoing, visualization of amyloid-associated pathologies in mice by PET would open a new avenue for monitoring dynamic status of amyloid deposition in living brains with minimal interference. Additional major benefit of PET imaging is also offered by the flexibility in designing imaging probes for specific purposes, allowing us to target different molecules of interest in the same individuals. This is of pivotal importance in mechanistic evaluation of amyloid β peptide (Aβ) immunization and other related anti-amyloid treatments (Dodel et al., 2003).

We have found that a PET ligand for peripheral benzodiazepine receptor (PBR), more specifically N-(5-fluoro-2-phenoxyphenyl)-N-(2-[¹⁸F]fluoroethoxy-5-methoxybenzyl)acetamide, termed [¹⁸F]fluoroethyl(FE)-DAA1106, which we recently developed for capturing glial activation (Zhang et al., 2004), can be used, preferebly in combination with amyloid probes, to longitudinally assess contribution of neuroinflammation to therapeutic and adverse effects. Thus, according to an embodiment of the present invention, the following method is provided:

a method for monitoring a therapy on a mammal having a neurodegenerative or neuroinflammatory disorder, comprising the steps of:

a) imaging the mammal using a radio-labeled PBR ligand;

b) administrating in the mammal at least one anti-amyloid or anti-neuroinflammatory agent;

c) imaging the mammal of the step b) using a radio-labeled PBR ligand; and

d) detecting the level of central nervous system (CNS) neuroinflammation by the signals from the radio-labeled PBR ligand.

The steps a), b), and/or c) may be repeated as necessary.

According to another embodiment of the present invention, the following method is provided:

a method for monitoring the response to a therapy in a mammal having a neurodegenerative or neuroinflammatory disorder that obtains or has obtained a therapy for that neurodegenerative or neuroinflammatory disorder, comprising the steps

a) imaging the mammal using a radio-labeled PBR ligand before therapy,

b) imaging the mammal of step a) using a radio-labeled PBR ligand,

c) comparing the level of CNS neuroinflammation using the signals obtained by the radio-labeled PBR ligand.

The steps a) and/or b) may be repeated as necessary.

According to another embodiment of the present invention, the following method is provided:

a method for monitoring a response to a therapy for a neurodegenerative or neuroinflammatory disorder on a mammal having the disorder, comprising the steps of:

a) administering a radio-labeled PBR ligand to the mammal to image the mammal; and

b) detecting the level of CNS neuroinflammation using the signal from the radio-labeled PBR ligand.

The step a) may be repeated as necessary, and the signals from the radio-labeled peripheral benzodiazepine receptor ligand may be compared to each other.

A another embodiment of the present invention relates to use of a radio-labeled PBR ligand, preferably [¹⁸F]FE-DAA1106, for the preparation of a composition useful for administration to a patient for the monitoring of the therapy of neurodegenerative or neuroinflammatory disorders.

A still another embodiment of the present invention relates to a radio-labeled PBR ligand or composition comprising the ligand, or a kit or system comprising the ligand for monitoring a response to a therapy of a neurodegenerative or neuroinflammatory disease.

According to a preferred embodiment, the diseases include Alzheimer's disease and multiple Sclerosis. The radio-labeled PBR ligand is preferably [¹⁸F]FE-DAA1106. The mammal can be a human being.

A still another embodiment of the present invention relates to a method for identifying an agent useful for treating a mammal having a disease associated with aggregated amyloid, comprising the steps:

a) administering an agent of interest to a non-human mammal;

b) imaging the non-human mammal by a radio-labeled PBR ligand, preferably [¹⁸F]FE-DAA1106;

d) repeating the steps a) and b) as necessary; and

d) selecting the agent which improves a neuroinflammatorial state of the mammal on the basis of the signal from the radio-labeled PBR receptor ligand.

A still another embodiment of the present invention relates to an agent identified by the method as mentioned above.

Administering compound(s) means administering via any route known to the person skilled in the art and includes but is not limited to oral administration or administration by injection. Injection might be intravenously, parenteral or subcutaneously.

### BRIEF DESCRIPTION OF THE DRAWINGS

FIG. 1. FIGS. 1 (A)-(I) are photographs. Amyloid elimination and glial activation during the course of anti-amyloid treatment as visualized by longitudinal PET scans. (A and B) PET maps of [¹⁸F]FE-DAA1106 (B) in a 20-month-old APP Tg mouse (Tg #3), generated by averaging dynamic data at 0-60 min (B), and superimposed on MRI template. Images were obtained before (PRE; left panel) and 1 (middle panel) and 2 (right panel) weeks after passive Aβ immunization. Vehicle alone and anti-Aβ antibody were injected into the left and right hippocampi, respectively. (D) Ratio between [¹⁸F]FE-DAA1106 radioactivities (at 0-60 min after the radiotracer administration) in antibody—and vehicle-injected hippocampi, showing markedly elevated neuroinflammatory response triggered by antibody injection (left panel; F_{(2, 4)}=16.7 and p<0.05 for main effect of time by repeated-measures ANOVA) and close correlation between levels of neuroinflammation and amyloid at 1 and 2 weeks after treatment (right panel; R²=0.942, p<0.01 by t-test). Solid line represents regression. (E-H) Double fluorescence labeling of amyloid (FSB; E and F) and microglia (Iba-1; G and H) in the left (E and G) and right (F and H) hippocampi of a Tg mouse (Tg #1) at 2 weeks after immunization. (I) Load of FSB-positive amyloid in the hippocampus, indicating a significant left-right difference (p<0.05 by t-test). Horizontal bars in graphs represent mean values.

### BEST MODE FOR CARRYING OUT THE INVENTION

The aim of this study was to prove the power of animal PET technology in pursuit of amyloidogenesis and evaluation of emerging anti-amyloid treatments. Two independent groups demonstrated that [¹¹C]6-OH-BTA-1-PET data in brains of mice developing abundant plaque lesions were virtually indistinguishable from those in wild-type (WT) mouse brains (Klunk et al., 2005; Toyama et al., 2005). A possible reason for the insensitivity of PET imaging in capturing mouse amyloid may lie in the paucity of high-affinity binding sites for the radioligand in APP Tg mouse brains when compared with AD brains (Klunk et al., 2005). Thus, we have overcome this problem by visualizing neuroinflammatory changes intimately associated with amyloidosis, by using a specific PBR radioligand, [¹⁸F]FE-DAA1106. Furthermore, advantages of in vivo PET measurement of amyloid have been reinforced by paralleling assays using amyloid radioligand and [¹⁸E]FE-DAA1106 to follow the course of Aβ immunization.

Examples as mentioned below are to explain the present invention in detail, and the present invention should not be limited at all by them.

### Example 1

### 1. Materials and Methods

Animals. The animals were maintained and handled in accordance with the recommendations of the US National Institutes of Health and institutional guidelines at the National Institute of Radiological Sciences. All animal experiments conducted here were approved by the Animal Ethics Committee of the National Institute of Radiological Sciences.

Tg mice termed APP23 mice, which overexpress the Swedish doubly mutant APP751 under the control of a neuron-specific Thy-1 promoter element, were generated as described in detail previously (Sturchler-Pierrat et al., 1997). The strain was maintained on C57BL/6J background, and female mice were employed for the experiments. Female non-Tg littermates were also used as WT controls.

Generation of MRI template. A 12-month-old C57BL/6J mouse was lethally anesthetized by pentobarbital. The mouse head was embedded in 3% aqueous agarose, and scanned by 9.4-Tesla Bruker AVANCE 400WB imaging spectrometer (Bruker BioSpin, Ettlingen, Germany), as described previously (Higuchi et al., 2005). Coronal T2-weighted MR images were acquired by using a 3-D fast spin-echo sequence with the following imaging parameters: TE=5.5 ms, TR=3,000 ms, RARE factor=32, field of view (FOV)=20×20×25 mm³, matrix dimensions=256×512×60, and nominal resolution=78 μm×39 μm×417 μm. The MRI data were used as an anatomical template for the subsequent PET studies.

[¹⁸F]FE-DAA1106, a PET ligand for PBR, was radiosynthesized using its desmethyl precursor, DAA1123 (generously provided by Taisho Pharmaceutical, Tokyo, Japan), as described elsewhere in detail (Zhang et al., 2004). The radiochemical purity of the end product exceeded 95%, and the specific radioactivity was 120±20.5 GBq/μmol at the end of synthesis.

Small animal PET imaging. All PET scans were performed using microPET Focus 220 animal scanner (Siemens Medical Solutions USA, Knoxville, Tenn.) designed for rodent and small monkeys, which provides 95 transaxial slices 0.815 mm (center-to-center) apart, a 19.0-cm transaxial FOV and a 7.6-cm axial FOV (Tai et al., 2005). Prior to the scans, the mice were anesthetized with 1.5% (v/v) isoflurane. After transmission scans for attenuation correction using a ⁶⁸Ge-⁶⁸Ga point source, emission scans were acquired for 60 min in a 3D list mode with an energy window of 350-750 keV, immediately after the intravenous injection of [¹¹C]6-OH-BTA-1 (30.0±6.8 MBq) or [¹⁸F]FE-DAA1106 (15.3±4.6 MBq). All list-mode data were sorted into 3D sinograms, which were then Fourier rebinned into 2D sinograms (frames, 10×1, 8×5 and 1×10 min). Dynamic images were reconstructed with filtered back-projection using a 0.5-mm Hanning's filter. Volumes of interest (VOIs) were placed on multiple brain areas using PMOD® image analysis software (PMOD Group, Zurich, Switzerland) with reference to the MRI template.

To assess capability of the present imaging system in monitoring effects of anti-amyloid treatment, we scanned Tg mice at multiple time points during the time course of passive Aβ immunization. Intrahippocampal injection of anti-Aβ antibody was performed based on established procedures (Wilcock et al., 2003). Three Tg mice aged 20, 21 and 24 months were anesthetized with 1.5% (v/v) isofurane, and placed in a stereotactic frame (Narishige, Tokyo, Japan). Using a 30-gauge needle connected to a 10-μl Hamilton syringe, 1 μl of mouse monoclonal antibody against amino-terminal portion of Aβ (6E10; Signet Laboratories, Dedham, Mass.; 1 mg/ml) and vehicle alone were injected into the right and left hippocampi, respectively (stereotactic coordinates:
anteroposterior, −2.8 mm; mediolateral, 2.0 mm; and dorsoventral, 3.0 mm from the bregma), over 2 min. The needle was thereafter raised by 1 mm, and injection of 1 μl solution was repeated. Total 3 PET scans using [¹⁸F]FE-DAA1106 were performed for each mouse at 1 or 2 weeks before and 1 and 2 weeks after the antibody injection. Mouse brains were thereafter dissected, and histochemically examined with FSB and rabbit polyclonal antibody against ionized calcium binding adapter molecule 1 (Iba-1; Wako Pure Chemicals, Osaka, Japan) recognizing microglia.

Statistical analyses. All statistical examinations in the present study were performed by SPSS software (SPSS, Chicago, Ill.). For comparisons of radiotracer uptake among regions and between WT and Tg mice, we performed 2-way repeated-measures analysis of variance (ANOVA). Correlations of radiotracer uptake with age and amyloid load were tested by the t-statistic.

### 2. Results

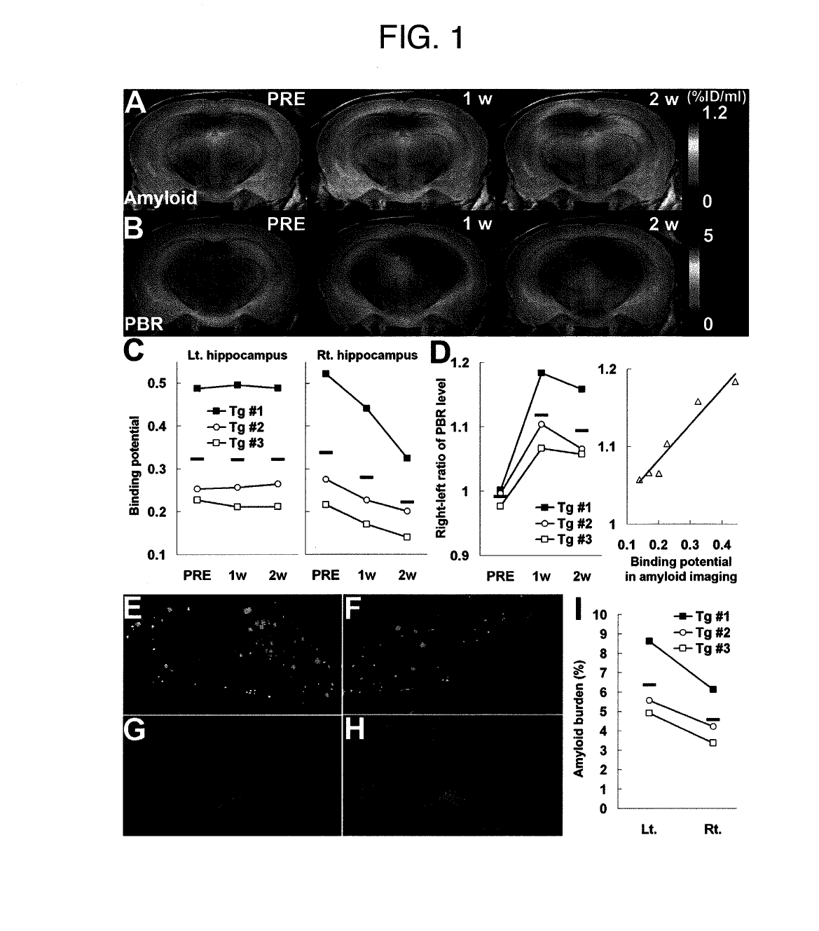

The potential utility of the present imaging system in assessing amyloid levels along the time course of anti-amyloid treatment was supported by our multi-scan, PET analysis of Tg mice before and 1 and 2 weeks after intrahippocampal injection of anti-Aβ antibody for the purpose of passive Aβ immunization (Wilcock et al., 2003). PET scans of the same individual clearly indicate prominent neuroinflammation induced by injected antibody, as monitored by PET with [¹⁸F]FE-DAA1106 (FIG. 1B). The right-left ratio of PBR level indicated marked activation of glial cells in the antibody-treated hippocampus (left panel in FIG. 1D). Significantly, the magnitude of neuroinflammatory responses to antibody injection was well correlated with the amount of amyloid (right panel in FIG. 1D). Therapeutic efficacy of Aβ immunization was confirmed by direct microscopic examination of dissected brains, as marked reduction of amyloid load (FIGS. 1E, 1F, 1I) and increase of hypertrophic microglia (FIGS. 1G, 1H) were demonstrated. Difference in mean value of amyloid burden between the antibody-injected and untreated hippocampi was 28.1%.

### 3. Discussion

The present work provides the first explicit evidence that an imaging probe, which has been applied in humans, is capable of noninvasively visualizing amyloid-related neuroinflammation in living animal models. This permits a comparative evaluation of amyloidogenic processes in humans and mice using the same quantitative indices, and thus assists mechanistic understanding of amyloid pathogenesis in both species. In addition, the utility of longitudinal PET study in quantitatively assessing alterations of amyloid levels as a function of age and in response to treatment is demonstrated for the first time, proving technological significance of the present achievement particularly in search of objective diagnostic and outcome measures for preclinical and clinical researches.

Because PET measurements require a very small amount of imaging agent relative to nonradioactive approaches, our current methodology offer a safe tool to monitor brain amyloid in mice without overt toxicity. This advantage is also of particular significance as prominent pharmacological effects of injected amyloid-binding tracers on the formation of amyloid (Lee, 2002; Masuda et al., 2006) are unlikely in PET studies. The present observations suggest that PET imaging of amyloid-related neuroinflammation permits robust preclinical evaluation of therapeutic strategies modifying pathological course of AD, and potentially provides a quantitative outcome measure in clinical trials of these treatments.

As evidenced here, the benefits of multi-scan, PET study in the same individual include a high statistical power, and analysis of 3 Tg mice indeed was sufficient to statistically examine effects of Aβ immunization on inflammatory response (FIGS. 1C, 1D). Moreover, the magnitude of glial activation after immunization is closely associated with the amount of Aβ amyloid. Excessive neuroinflammation may induce neurotoxic insults, as exemplified by occurrence of meningoencephalitis in those who received Aβ vaccination (Orgogozo et al., 2003; Nicoll et al., 2003). Additionally, our recent investigation on a mouse model of neurofibrillary tangles using tritiated DAA1106 has indicated that microglial overactivation in AD and other tauopathy brains could lead to accelerated tau pathogenesis and neuronal loss (Yoshiyama et al., 2007). Hence, the present result implies need for initiating therapeutic intervention at an unadvanced stage of amyloid pathology to minimize adverse effects, and supports the utility of [¹⁸F]FE-DAA1106 in conjunction with an amyloid radioligand in optimizing treatment protocols.

Notwithstanding several technical aspects to be further improved, such as spatial resolution of the scanner (˜1.5 mm) (Tai et al., 2005), our results rationalize the use of micro PET for elucidating molecular regulators of amyloid deposition and for proving mechanistic concepts of emerging approaches to therapeutic interventions (Scarpini et al., 2003; Dodel et al., 2003). This in vivo system also offers an efficient strategy to preclinically compare pharmacokinetic properties of multiple candidate amyloid probes in the same individual. In such a study, the distinct nature of amyloid aggregates in humans and mice is likely overcome by sensitively capturing the high-affinity components in mouse plaque using high-specific radioactivity ligands, providing extrapolatability of the finding in mice to humans.

### REFERENCES

Bacskai B J, Hickey G A, Skoch J, Kajdasz S T, Wang Y, Huang G F, Mathis C A, Klunk W E, Hyman B T (2003) Four-dimensional multiphoton imaging of brain entry, amyloid binding, and clearance of an amyloid-beta ligand in transgenic mice. Proc Natl Acad Sci USA 100:12462-12467.

Cummings J L, Cole G (2002) Alzheimer disease. JAMA 287:2335-2338.

Dodel R C, Hampel H, Du Y (2003) Immunotherapy for Alzheimer's disease. Lancet Neurol 2:215-220.

Engler H, Forsberg A, Almkvist O, Blomquist G, Larsson E, Savitcheva I, Wall A, Ringheim A, Langstrom B, Nordberg A (2006) Two-year follow-up of amyloid deposition in patients with Alzheimer's disease. Brain 129:2856-2866.

Higuchi M, Iwata N, Matsuba Y, Sato K, Sasamoto K, Saido T C (2005) ¹⁹F and ¹H MRI detection of amyloid b plaques in vivo. Nat Neurosci 8:527-533.

Hintersteiner M, Enz A, Frey P, Jaton A L, Kinzy W, Kneuer R, Neumann U, Rudin M, Staufenbiel M, Stoeckli M, Wiederhold K H, Gremlich H U (2005) In vivo detection of amyloid-beta deposits by near-infrared imaging using an oxazine-derivative probe. Nat Biotechnol 23:577-583.

Hsiao K, Chapman P, Nilsen S, Eckman C, Harigaya Y, Younkin S, Yang F, Cole G. (1996) Correlative memory deficits, Aβ elevation, and amyloid plaques in transgenic mice. Science 274:99-102.

Ichise M, Liow J S, Lu J Q, Takano A, Model K, Toyama H, Suhara T, Suzuki K, Innis R B, Carson R E (2003) Linearized reference tissue parametric imaging methods: application to [¹¹C]DASB positron emission tomography studies of the serotonin transporter in human brain. J Cereb Blood Flow Metab 23:1096-1112.

Klunk W E, Engler H, Nordberg A, Wang Y, Blomqvist G, Holt D P, Bergstrom M, Savitcheva I, Huang G F, Estrada S, Ausen B, Debnath M L, Barletta J, Price J C, Sandell J, Lopresti B J, Wall A, Koivisto P, Antoni G, Mathis C A, Langstrom B (2004) Imaging brain amyloid in Alzheimer's disease with Pittsburgh Compound-B. Ann Neurol 55:306-319.

Klunk W E, Lopresti B J, Ikonomovic M D, Lefterov I M, Koldamova R P, Abrahamson E E, Debnath M L, Holt D P, Huang G F, Shao L, DeKosky S T, Price J C, Mathis C A (2005) Binding of the positron emission tomography tracer Pittsburgh compound-B reflects the amount of amyloid-β in Alzheimer's disease brain but not in transgenic mouse brain. J Neurosci 25:10598-10606.

Lammertsma A A, Bench C J, Hume S P, Osman S, Gunn K, Brooks D J, Frackowiak R S (1996) Comparison of methods for analysis of clinical [¹¹C]raclopride studies. J Cereb Blood Flow Metab 16:42-52.

Lee V M (2002) Amyloid binding ligands as Alzheimer's disease therapies. Neurobiol Aging 23:1039-1042.

Lockhart A, Ye L, Judd D B, Merritt A T, Lowe P N, Morgenstern J L, Hong G, Gee A D, Brown J (2005) Evidence for the presence of three distinct binding sites for the thioflavin T class of Alzheimer's disease PET imaging agents on β-amyloid peptide fibrils. J Biol Chem 280:7677-7684.

Masuda M, Suzuki N, Taniguchi S, Oikawa T, Nonaka T, Iwatsubo T, Hisanaga S, Goedert M, Hasegawa M (2006) Small molecule inhibitors of α-synuclein filament assembly. Biochemistry 45:6085-6094.

Mintun M A, Larossa G N, Sheline Y I, Dence C S, Lee S Y, Mach R H, Klunk W E, Mathis C A, DeKosky S T, Morris J C (2006) [¹¹C]PIB in a nondemented population: potential antecedent marker of Alzheimer disease. Neurology 67:446-452.

Nichols L, Pike V W, Cai L, Innis R B (2006) Imaging and in vivo quantitation of beta-amyloid: an exemplary biomarker for Alzheimer's disease? Biol Psychiatry 59:940-947.

Nicoll J A, Wilkinson D, Holmes C, Steart P, Markham H, Weller R O (2003) Neuropathology of human Alzheimer disease after immunization with amyloid-β peptide: a case report. Nat Med 9:448-452.

Noguchi J, Suzuki K (2003) Automated synthesis of the ultra high specific activity of [¹¹C]Ro15-4513 and its application in an extremely low concentration region to an ARG study. Nucl Med Biol 30:335-343.

Orgogozo J M, Gilman S, Dartigues J F, Laurent B, Puel M, Kirby L C, Jouanny P, Dubois B, Eisner L, Flitman S, Michel B F, Boada M, Frank A, Hock C. (2003) Subacute meningoencephalitis in a subset of patients with AD after Aβ42 immunization. Neurology 61:46-54.

Price J C, Klunk W E, Lopresti B J, Lu X, Hoge J A, Ziolko S K, Holt D P, Meltzer C C, DeKosky S T, Mathis C A (2005) Kinetic modeling of amyloid binding in humans using PET imaging and Pittsburgh Compound-B. J Cereb Blood Flow Metab 25:1528-1547.

Sair H I, Doraiswamy P M, Petrella J R (2004) In vivo amyloid imaging in Alzheimer's disease. Neuroradiology 46:93-104.

Sato K, Higuchi M, Iwata N, Saido T C, Sasamoto K (2004) Fluoro-substituted and ¹³C-labeled styrylbenzene derivatives for detecting brain amyloid plaques. Eur J Med Chem 39:573-578.

Scarpini E, Scheltens P, Feldman H (2003) Treatment of Alzheimer's disease: current status and new perspectives. Lancet Neurol 2:539-547.

Shoghi-Jadid K, Small G W, Agdeppa E D, Kepe V, Ercoli L M, Siddarth P, Read S, Satyamurthy N, Petric A, Huang S C, Barrio J R (2002) Localization of neurofibrillary tangles and beta-amyloid plaques in the brains of living patients with Alzheimer disease. Am J Geriatr Psychiatry 10:24-35.

Sturchler-Pierrat C, Abramowski D, Duke M, Wiederhold K H, Mistl C, Rothacher S, Ledermann B, Bürki K, Frey P, Paganetti P A, Waridel C, Calhoun M E, Jucker M, Probst A, Staufenbiel M, Sommer B (1997) Two amyloid precursor protein transgenic mouse models with Alzheimer disease-like pathology. Proc Natl Acad Sci USA 94:13287-13292.

Suzuki K, Inoue O, Hashimoto K, Yamasaki T, Kuchiki M, Tamate K (1985) Computer-controlled large scale production of high specific activity [¹¹C]RO 15-1788 for PET studies of benzodiazepine receptors. Int J Appl Radiat Isot 36:971-976.

Tai Y C, Ruangma A, Rowland D, Siegel S, Newport D F, Chow P L, Laforest R (2005) Performance evaluation of the microPET focus: a third-generation microPET scanner dedicated to animal imaging. J Nucl Med 46:455-463.

Toyama H, Ye D, Ichise M, Liow J S, Cai L, Jacobowitz D, Musachio J L, Hong J, Crescenzo M, Tipre D, Lu J Q, Zoghbi S, Vines D C, Seidel J, Katada K, Green M V, Pike V W, Cohen R M, Innis R B (2005) PET imaging of brain with the β-amyloid probe, [¹¹C]6-OH-BTA-1, in a transgenic mouse model of Alzheimer's disease. Eur J Nucl Med Mol Imaging 32:593-600.

Verhoeff N P, Wilson A A, Takeshita S, Trop L, Hussey D, Singh K, Kung H F, Kung M P, Houle S (2004) In-vivo imaging of Alzheimer disease β-amyloid with [¹¹C]SB-13 PET. Am J Geriatr Psychiatry 12:584-595.

Wilcock D M, DiCarlo G, Henderson D, Jackson J, Clarke K, Ugen K E, Gordon M N, Morgan D (2003) Intracranially administered anti-Aβ antibodies reduce β-amyloid deposition by mechanisms both independent of and associated with microglial activation. J Neurosci 23:3745-3751.

Ye L, Morgenstern J L, Gee A D, Hong G, Brown J, Lockhart A (2005) Delineation of positron emission tomography imaging agent binding sites on β-amyloid peptide fibrils. J Biol Chem 280:23599-23604.

Yoshiyama Y, Higuchi M, Zhang B, Huang S M, Iwata N, Saido T C, Maeda J, Suhara T, Trojanowski J Q, Lee V M (2007) Synapse loss and microglial activation precede tangles in a P301S tauopathy mouse model. Neuron 53:337-351.

Zhang M R, Maeda J, Ogawa M, Noguchi J, Ito T, Yoshida Y, Okauchi T, Obayashi S, Suhara T, Suzuki K (2004) Development of a new radioligand, N-(5-fluoro-2-phenoxyphenyl)-N-(2-[¹⁸F]fluoroethyl-5-methoxybenzyl)acetamide, for pet imaging of peripheral benzodiazepine receptor in primate brain. J Med Chem 47:2228-2235.

Zhang M R, Suzuki K (2005) Sources of carbon which decrease the specific activity of [¹¹C]CH₃I synthesized by the single pass I₂ method. Appl Radiat Isot 62:447-450.

The contents of the references as mentioned above are hereby incorporated by reference into this application.

### INDUSTRIAL APPLICABILITY

We provide the first evidence for capability of a high-resolution positron emission tomographic (PET) imaging system in quantitatively mapping amyloid-related neuroinflammation in living amyloid precursor protein transgenic (Tg) mice. Neuroinflammatory responses induced by anti-amyloid treatment using antibody against amyloid β peptide were successfully monitored by multiple PET scans with [¹⁸F]FE-DAA1106 along the time course of treatment, and were found to be closely correlated with levels of amyloid.

Our results support the usefulness of the small animal-dedicated PET system in conjunction with appropriate Tg model for not only clarifying mechanistic properties of amyloidogenesis in mouse models but also preclinical tests of emerging diagnostic and therapeutic approaches to Alzheimer's disease.

1. A method for monitoring a response to a therapy on a mammal having a neurodegenerative or neuroinflammatory disorder, comprising the steps of:
a) imaging the mammal using a radio-labeled PBR ligand;
b) administrating in the mammal at least one anti-amyloid or anti-neuroinflammatory agent;
c) imaging the mammal of step b) using a radio-labeled PBR ligand; and
d) detecting the level of CNS neuroinflammation by the signals from the radio-labeled PBR ligand.

2. The method according to claim 1, wherein the steps a), b), and/or c) are repeated as necessary.

3. A method for monitoring a response to a therapy for a neurodegenerative or neuroinflammatory disorder on a mammal having the disorder, comprising the steps of:
a) imaging the mammal using a radio-labeled PBR ligand before the therapy;
b) imaging the mammal of step a) using a radio-labeled PBR ligand after the therapy; and
c) detecting the level of CNS neuroinflammation using the signals from the radio-labeled peripheral PBR ligand.

4. The method according to claim 3, wherein the steps a) and/or b) are repeated as necessary.

5. A method for monitoring a response to a therapy for a neurodegenerative or neuroinflammatory disorder on a mammal having the disorder, comprising the steps of:
a) administering a radio-labeled PBR ligand to the mammal to image the mammal; and
b) detecting the level of CNS neuroinflammation using the signal from the radio-labeled PBR ligand.

6. The method according to claim 5, wherein the step a) is repeated as necessary.

7. The method according to claim 6, wherein the signals are compared to each other.

8. The method according to claim 1, wherein the radio-labeled PBR ligand is N-(5-fluoro-2-phenoxyphenyl)-N-(2-[¹⁸F]fluoroethoxy-5-methoxybenzyl)acetamide, termed [¹⁸F]FE-DAA1106.

9. The method according to claim 1, wherein the disease is Alzheimer's disease.

10. The method according to claim 1, wherein the disease is Multiple Sclerosis.

11. (canceled)

12. (canceled)

13. (canceled)

14. A radio-labeled PBR ligand or a composition comprising the ligand for monitoring a response to a therapy of neurodegenerative or neuroinflammatory disorder.

15. The ligand or composition according to claim 14, wherein the disorder is Alzheimer's disease or multiple Sclerosis.

16. The ligand or composition according to claim 14, wherein the radio-labeled PBR ligand is [¹⁸F]FE-DAA1106.

17. A kit or system comprising a radio-labeled PBR ligand for monitoring a therapy of neurodegenerative or neuroinflammatory disorders.

18. The kit or system according to claim 17, wherein the disorder is Alzheimer's disease or multiple Sclerosis.

19. The kit or system according to claim 17, wherein the radio-labeled PBR ligand is [¹⁸F]FE-DAA1106.

20. A method for identifying an agent useful for treating a mammal having a disease associated with aggregated amyloid, comprising the steps:
a) administering an agent of interest to a non-human mammal;
b) imaging the non-human mammal by a radio-labeled PBR ligand;
c) repeating the steps a) and b) as necessary; and
d) selecting the agent which improves a neuroinflammatorial state of the mammal on the basis of the signal from the radio-labeled PBR ligand.

21. The method according claim 20 wherein the disease is Alzheimer's disease.

22. A method according to claim 20, wherein the radio-labeled PBR ligand is [¹⁸F]FE-DAA1106.

23. An agent identified by a method according to claim 20.
